(19) United States
(12) Patent Application Publication
(10) Pub. No.: US 2011/0155141 A1
(43) Pub. Date: Jun. 30, 2011

(54) Wearable Article That Stiffens Upon Sudden Force

(76) Inventors: Lawrence H. Sawyer, Neenah, WI (US); Eric D. Johnson, Larsen, WI (US); James D. McManus, Hortonville, WI (US); John Gavin MacDonald, Decatur, GA (US); Russell F. Ross, Atlanta, GA (US)

(21) Appl. No.: 12/975,021

(22) Filed: Dec. 21, 2010

Related U.S. Application Data
(63) Continuation-in-part of application No. 12/647,613, filed on Dec. 28, 2009.

(51) Int. Cl. A61F 13/00

(52) U.S. Cl. 128/846

(57) ABSTRACT

A wearable article made from a nonwoven fabric that includes a plurality of coated fibers. The coated fibers have an exterior surface and a coating composition on at least a portion of the exterior surface. The coating composition includes an aminofunctionalized silane and a dialdehyde, where the weight percent of the dialdehyde in the coating composition is greater than the weight percent of the silane in the coating composition. The wearable article can be a wrap, brace, support, compression hosiery, bandage or compress. When worn, the wearable article is initially flexible but becomes rigid and stiff when the coated fibers experience a sudden force or impact. The wearable article does not include a fluid.

This application is a continuation-in-part application claiming priority from presently co-pending U.S. application Ser. No. 12/647,613 entitled “Puncture Resistant Fabric” filed on Dec. 28, 2009, in the names of John Gavin MacDonald et al.

### FIELD OF THE INVENTION

The present invention relates to a wearable article that includes a nonwoven fabric. The nonwoven fabric includes coated fibers. The exterior surface of the fibers includes a coating composition that includes an aminofunctionalized silane and dialdehyde. The weight percent of dialdehyde in the coating composition is greater than the weight percent of the silane in the coating composition. The coating composition on the fibers causes the nonwoven fabric to change from a flexible condition to a stiffened, rigid condition when the nonwoven fabric experiences a sudden force.

### BACKGROUND OF THE INVENTION

The present invention relates to wearable articles that can be worn to protect parts of the body that are susceptible to injury from unexpected falls or other events that cause parts of the body to experience sudden force. While anyone could experience a sudden, unexpected fall, some population segments such as the elderly or physically compromised are potentially more susceptible. Conditions that could increase the likelihood of an unexpected fall or similar event include the type of clothing or shoes being worn or the condition of the surface across which a person is traveling, such as an ice-covered sidewalk, a wet floor or an irregular surface (e.g. cobblestone).

An unexpected fall can lead to trauma/injury to almost any part of the body including hands, wrists, elbows, shoulders, neck, back, hips, knees and ankles. The treatment and recovery from injuries to any of these body parts can be painful, lengthy and costly. A person suffering from such an injury could entirely lose use of or experience limited use of the injured body part for several weeks or even several months depending on the severity of the injury and the person's condition at the time of the injury. The public health impact of hip fractures is described in U.S. Pat. No. 5,599,290 issued to Hayes et al. (hereinafter “the '290 patent”). The '290 patent relates to a garment for reducing the risk of bone fracture of a human or animal caused by impact forces on a vulnerable region having a bone part near the skin surface where the vulnerable region is proximate to a soft tissue region. The garment has an arrangement for shunting a substantial portion of the impact energy from the vulnerable region to the soft tissue region. The garment of the '290 patent includes a dilatant material that is relatively stiff near the time of impact and relatively fluid at other times.

A variety of products for supporting/bracing vulnerable or injured parts of the body are already commercially available. For example, the 3M Company manufactures and sells a line of personal care products under the ACE brand; products sold under the ACE brand include braces, supports, compression hosiery, elastic bandages and cold/hot compresses. Additionally, flexible wraps that deliver heat/cold therapy to a body part are known; for example, U.S. patent application Ser. No. 10/645,447 describes such flexible wraps. Further, in addition to the bone fracture prevention garment of the '290 patent, body armor, such as armor that may be worn by police and military professionals, that has shear-thickening fluids incorporated into the fabric forming the body armor is also known. Such body armor is described in U.S. Pat. No. 7,226,878 issued to Wagner et al. (hereinafter “the '878 patent”). In both the bone fracture prevention garment of the '290 patent and the body armor of the '878 patent, the garment/fabric includes a dilatent material/shear-thickening fluid that is in a fluid state until a sudden force/impact is experienced by the user of the garment/body armor. When a sudden force/impact occurs, the dilatent material/shear-thickening fluid absorbs and dissipates kinetic energy and becomes rigid and stiff.

Wearable articles such as wraps, braces, supports, compression hosiery, bandages and compresses can be made of nonwoven fabrics. Nonwoven fabrics or webs are cost-advantaged in these types of applications. As used herein, the term “nonwoven fabric or web” generally refers to a web having a structure of individual fibers or threads which are interlaid, but not in an identifiable manner as in a knitted fabric. Examples of suitable nonwoven fabrics or webs include, but are not limited to, meltblown webs, spunbond webs, carded webs, etc. The basis weight of the nonwoven web may generally vary, such as from about 0.1 grams per square meter (“gsm”) to about 120 gsm or more. Nonwoven fabrics are capable of providing several benefits including breathability, drapability and comfort. A nonwoven laminate such as a spunbond-meltblown-spunbond (SMS) laminate may be useful and cost-effective for forming wearable articles such as wraps, braces, supports, compression hosiery, bandages and compresses. SMS laminates generally include nonwoven outer layers of spunbonded polyolefins and an inner barrier layer of meltblown polyolefin. As used herein, the term “meltblown web” generally refers to a nonwoven web that is formed by a process in which a molten thermoplastic material is extruded through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Generally speaking, meltblown fibers may be microfibers that are substantially continuous or discontinuous, generally smaller than 10 microns in diameter, and generally tacky when deposited onto a collecting surface. As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is widely known. Spunbond fibers are generally not tacky when they are deposited onto a collecting surface and may have diameters less than about 40 microns, and are often between about 5 to about 20 microns.

Given the potential usefulness of products like wraps, braces, supports, compression hosiery, bandages and compresses in protecting parts of the body vulnerable to injury, there remains a need for a wearable article that is breathable and flexible during ordinary wear, but that becomes rigid and stiff upon experiencing a sudden force or impact. Additionally, there remains a need for such a wearable article that has the property and characteristic of changing its physical state without using a component that is initially in a fluid state. Further, there remains a need for a dry, cured (i.e. not fluid or liquid) component that will not leach or migrate out of the wearable article.

### SUMMARY OF THE INVENTION

The present invention is directed, in part, to a wearable article that may be worn by a human or an animal to prevent injury or trauma to a body part susceptible to an unexpected, sudden force such as may occur during a fall. The wearable article may be a wrap, brace, support, compression hosiery, bandage or compress. The wearable article includes a nonwoven fabric formed of a plurality of fibers. The fibers are generally cylindrical in shape and therefore, define an exterior surface. A coating composition is disposed on at least a portion of the exterior surfaces of the fibers. In certain aspects, at least about 50% of the exterior surfaces of the fibers are coated with the coating composition. In some aspects, at least about 75% and, in particular aspects, at least about 90% of the exterior surfaces of the fibers may be coated with the coating composition. The fibers may also be corona treated to enhance application of the coating composition to the fibers. The coating composition modifies the mechanical properties of the fibers and, therefore, of the nonwoven fabric used to form the wearable article. In an initial state, when the wearable article is donned, the nonwoven fabric is flexible and drapable. If the body part that the wearable article is protecting experiences a sudden, perhaps unexpected, force, the nonwoven fabric will become rigid and stiff. The effect is that the wearable article assists with immobilizing the affected body part, thereby decreasing the risk of further injury or complications. The nonwoven fabric responds in this manner due to a change in mechanical properties caused by the effect of the sudden force on the coating composition that the nonwoven fabric fibers are coated with. The coating composition is “dry” and is not in a fluid state on the fibers forming the nonwoven fabric. The mechanical property change from a flexible, drapable fabric to a rigid, stiff fabric can happen in response to different types of forces including flexural, torsional, compressional, expansional, shear and impact forces.

The coating composition includes an aminofunctionalized silane and a dialdehyde, such as glutaraldehyde. The weight percent of dialdehyde in the coating composition is greater than the weight percent of aminofunctionalzied silane in the coating composition. In certain aspects, the weight percent of dialdehyde is at least twice the weight percent of aminofunctionalized silane, and may be at least four times the weight percent of aminofunctionalized silane in the coating composition. In particular aspects, aminopropyltriethoxysilane (APTES) or hexamethyldisilazane (HDMS) may be utilized as the aminofunctionalized silane, although other aminofunctionalized silanes are also suitable.

The wearable articles of the invention may be formed of nonwoven fabric that is air permeable or breathable and may be formed from any of a variety of nonwoven materials and processes. For example, the nonwoven fabric may be a laminate that includes a spunbond layer and a meltblown layer. The wearable article may also include a film material and the film material may be breathable. Prior to experiencing a sudden force, the nonwoven fabric is flexible and requires a total energy to compress that is at least 25% greater at a compression rate of 400 inches/minute compared to a total energy to compress at 200 inches/minute as measured by a ring compression test. After experiencing a sudden force, the nonwoven fabric becomes stiff and/or rigid; the total energy to compress of the nonwoven fabric after impact depends on the construction of the wearable article, the basis weight(s) of the nonwoven material(s) used and the type of coating composition applied to the fibers of the nonwoven material(s).

Because a purpose of the wearable articles of the invention is to reduce the risk of additional injury or trauma by assisting to immobilize the affected body part, it may also be beneficial for the wearable article to be able to deliver coldness to the affected body part in order to reduce swelling and/or inflammation. Alternatively, depending on the nature of the injury, it may be beneficial for the wearable article to provide heat to the affected body part to reduce soreness and/or stiffness. Because the wearable articles of the invention are intended to be relatively thin and comfortable to wear, the wearable article includes at least one generally planar surface. The planar surface may include a thermal change zone. The thermal change zone includes components to deliver coldness and/or heat, as desired. The thermal change zone may include two, planar reservoirs; each reservoir is configured to separately contain a thermal reagent capable of affecting a change in temperature when combined with another thermal reagent. The two, planar reservoirs may be separated by a frangible seal that is capable of rupturing when the wearable article experiences the sudden force. When the contents of the two reservoirs comingle, the temperature change occurs. For example, the thermal reagent in one of the reservoirs may be a proton-contributing material and the thermal reagent in the other reservoir may be a proton-accepting material. Alternatively, the thermal change zone may include a single reservoir that contains a thermal reagent that changes temperature when exposed to air. The thermal reagent would be exposed to air when the thermal change zone is exposed to a sudden force.

In another aspect of the present invention, a nonwoven fabric incorporated into the wearable article may include a plurality of coated fibers. Each coated fiber has an exterior surface and a coating composition disposed on at least a portion of the exterior surface of the fibers. The coating composition includes particles, an aminofunctionalized silane and a dialdehyde, such as glutaraldehyde. The weight percent of the dialdehyde in the coating composition is greater than the weight percent of the aminofunctionalized silane in the coating composition. In some aspects, the weight percent of dialdehyde in the coating composition is at least about twice the weight percent of aminofunctionalized silane in the coating composition. In some aspects, the weight percent of dialdehyde in the coating composition is at least twice the weight percent of particles in the coating composition. The particles may be silica, titanium dioxide, alumina or any one of a variety of other particles. While the size of the particles may vary greatly, the particles are preferably nanoparticles having an average particle size of less than 250 nanometers or, in selected aspects, less than 150 nanometers.

These aspects and additional aspects of the invention will be described in greater detail herein. Further, it is to be understood that both the foregoing general description and the following detailed description are exemplary and are intended to provide further explanation of the invention claimed.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a photomicrograph of a coated fiber formed in accordance with an aspect of the present invention;

FIG. 2 is a photomicrograph of a nonwoven fabric formed in accordance with one aspect of the present invention;

FIG. 3 is a representative drawing of a wearable article of the invention as worn around the ankle of a person who has been injured;

FIG. 4 is a representative drawing of a wearable article of the invention as worn around the ankle of a person who has been injured where the wearable article includes a thermal change zone with a planar reservoir;

FIG. 5 is a representative drawing of a wearable article of the invention as worn around the ankle of a person who has been injured in which the wearable article has a thermal change zone with two, planar reservoirs that are separated by a frangible seal; and

FIG. 6 is a representative drawing of a wearable article of the invention as worn around the ankle of a person who has been injured in which the wearable article has a thermal change zone with multiple planar reservoirs.

Repeat use of reference characters in the present specification and drawings is intended to represent same or analogous features or elements of the invention.

### DETAILED DESCRIPTION OF REPRESENTATIVE EMBODIMENTS

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is generally directed to wearable articles designed to be worn by humans or animals over body parts that are susceptible to injury or trauma because of experiencing a sudden, likely unexpected, force such as from a fall. The body parts that could be protected by the wearable articles of the invention include the neck, shoulders, elbows, wrists, back, hips, knees and ankles. The wearable articles of the invention include, but are not limited to wraps, braces, supports, compression hosiery, bandages or compresses. The present invention is generally directed to wearable articles that are formed entirely or in part of one or more nonwoven fabrics that include a plurality of coated fibers. The coating on the fibers causes the mechanical properties of the nonwoven fabric(s) to change when the wearable article and the body part it is protecting experience a sudden force. When the wearable article is initially donned, the nonwoven fabric is very flexible and drapable. Desirably, the wearable articles of the invention are relatively thin and generally planar so that when they are worn they are comfortable and do not interfere with how other garments fit the user. When the nonwoven fabric experiences a sudden force, it causes the nonwoven fabric to become rigid and stiff. The change to the mechanical properties of the nonwoven fabric results in the affected body part being partially or entirely immobilized from further motion that could potentially cause additional injury or trauma. The mechanical properties of the nonwoven fabric will change in response to sudden exposure to different types (i.e. direction) of forces including flexural, torsional, compressional, expansional, shear and impact forces.

The wearable articles of the invention can be formed from one or more types of nonwoven fabrics including spunbond webs, meltblown webs, bonded-carded webs and laminates of one or more nonwoven fabrics, such as SMS. The nonwoven fabrics may be extensible or elastic and the elasticity may be provided by elastic strands or elastic adhesive that is incorporated into the nonwoven fabric. Wearable articles that are elastic are capable of providing a close, snug fit to the targeted body part. Further, the wearable articles may include systems for fastening the wearable article around the targeted body part; such fastening systems include hook-and-loop fasteners, metal clips with prongs that engage the nonwoven fabric, snaps and other fasteners known in the art for securing wearable articles such as wraps, supports, braces and bandages. Alternatively, the nonwoven fabric may be self-adhering. Desirably, the coating of the nonwoven fabric fibers does not significantly interfere with the breathability of the nonwoven fabric. The wearable articles of the invention may also include a film material either separate from or in combination with the nonwoven fabric.

The plurality of fibers that form a nonwoven fabric are generally cylindrical in shape and therefore, have an exterior surface. The plurality of fibers forming the nonwoven fabrics/wearable articles of the invention have a coating composition disposed on at least a portion of the exterior surfaces of the fibers. The coating composition may be applied to the plurality of fibers forming the nonwoven fabric using traditional techniques such as dipping the nonwoven fabric in a vessel containing the coating composition and then squeezing excess coating composition off of the nonwoven fabric. The coating composition may also be sprayed or printed on the nonwoven fabric. After application and a reasonable drying period, the coating composition is in a dry (not liquid or fluid) state on the exterior surfaces of the plurality of fibers forming the wearable articles of the invention. While the coating composition of the present invention behaves similarly to dilatent/shear-thickening materials in response to a sudden force, the coating composition overcomes difficulties associated with fluid materials that might leach or migrate away from the nonwoven fabric. In one aspect, at least 50% of the exterior surfaces of the plurality of fibers may be coated with the coating composition. Additionally, at least 75% or at least 90% of the exterior surfaces of the plurality of fibers may be coated with the coating composition.

The coating composition of the invention includes an aminofunctionalized silane and a dialdehyde. The weight percent of the dialdehyde in the coating composition is greater than the weight percent of the aminiofunctionalized silane in the coating composition. More specifically, the weight percent of the dialdehyde in the coating composition is at least twice the weight percent of aminofunctionalized silane in the coating composition. In another aspect, the coating composition may include particles in addition to the aminofunctionalized silane and dialdehyde. In this aspect, the weight percent of dialdehyde in the coating composition is at least twice the weight percent of particles in the coating composition.

Many aminofunctionalize silanes are suitable for use in the coating composition of the present invention. For example, tetraethoxysilane (TEOS), having the formula Si(OC₂H₅)₄ is a suitable aminofunctionalized silane. TEOS can be used as a crosslinking agent in silicone polymers. 2-aminopropyltriethoxysilane (“APTES”) is an aminofunctionalized organosilane that is also suitable for use in the coating composition of the present invention. APTES provides superior bonds between inorganic substrates and organic polymers, and is represented by the chemical formula, NH₂CH₂CH₂CH₂Si(OC₂H₅)₃. Another suitable aminiofunctionalized silane, hexamethyldisilazane (HMDS), is a chemical compound with the formula HN[Si(CH₃)₃]₂. Other aminofunctionalized silanes include hexamethylsilazane and heptamethyldisilazane. Other suitable compounds include 3-aminopropyltriethoxysilane, bis[(3-triethoxysilyl)propyl]amine, 3-aminopropyltrimethoxysilane, 3-aminopropylmethyldiethoxysilane, 3-aminopropylmethyldimethoxysilane, aminoethylaminopropyltrimethoxysilane, aminoethylaminopropyltriethoxysilane, aminoethylaminopropylmethyldimethoxysilane, aminoethylaminopropylmethyldiethoxysilane, aminoethylaminomethyltriethoxysilane, aminoethylaminomethylmethyldiethoxysilane, diethylenetriaminopropyltrimethoxysilane, diethylenetriaminopropyltriethoxysilane, diethylenetriaminopropylmethyldimethoxysilane, diethylenetriaminopropylmethyldiethoxysilane, diethylenetriaminomethylmethyldiethoxysilane, (n-phenylamino)methyltrimethoxysilane, (n-phenylamino)methyltriethoxysilane, (n-phenylamino)methylmethyldimethoxysilane, (n-phenylamino)methylmethyldiethoxysilane, 3-(n-phenylamino)propyltrimethoxysilane, 3-(n-phenylamino)propyltriethoxysilane, 3-(n-phenylamino)propylmethyldimethoxysilane, 3-(n-phenylamino)propylmethyldiethoxysilane, diethylaminomethyltriethoxysilane, diethylaminomethyldiethoxysilane, diethylaminomethyltrimethoxysilane, diethylaminopropyltrimethoxysilane, diethylaminopropylmethyldimethoxysilane, diethylaminopropylmethyldiethoxysilane and n-(n-butyl)-3-aminopropyltrimethoxysilane.

The dialdehyde of the coating composition of the invention can be selected from alkyl or aromatic dialdehydes such as ethanedial (also known as glyoxal), butanedial (also known as succinaldehyde), pentanedial (also known as glutaraldehyde), and 1-4 benzenedicarboxaldehyde (also known as phthalic dicarboxaldehyde). Glutaraldehyde was selected as the dialdehyde compound to be utilized in the examples of the present invention. Glutaraldehyde is a colorless liquid with a pungent odor that has many uses such as crosslinking. In selected examples of the present invention, glutaraldehyde reacts with the aminofunctionalized silane to form a matrix. Glutaraldehyde was obtained from the Sigma-Aldrich Chemical Company (Milwaukee Wis.) and was used for each of the examples in Table 1.

In some aspects of the present invention, particles such as nanoparticles may be added to the silane and dialdehyde at any time during formation of the coating composition. As used herein, the term “nanoparticles” may include particles having an average diameter of less than about 1000 nanometers, although it is to be understood that larger particles may be useful in particular aspects of the present invention. The size of the nanoparticles will impact the ability of the nanoparticle to be adequately incorporated into the matrix of the coating composition. Although the size of the nanoparticles may be varied widely, the nanoparticle should be sufficiently small to enable its incorporation into the aminofunctionalized silane/dialdehyde network. In some aspects of the present invention, the nanoparticles may have an average diameter of less than 500 nm, and in other aspects less than 250 nm, while in selected aspects desirably less than 100 nm. The selection of the appropriate size of the particle for a particular application may also depend upon the desired rate of deformation (change in mechanical properties) of the coating composition.

The size of the nanoparticle that may be suitable for different aspects of the present invention may also depend, in part, on the nonwoven fabric that is coated for use in the wearable article. For example, large nanoparticles having an average diameter of greater than 400 nanometers may be suitable for use in a coating composition for a nonwoven fabric that has a very high level of breathability, a large void size and a large fiber size. Such a fabric may include one or two layers of a spunbond material having a basis weight in the range of about 0.5 to about 6.0 oz/yd² (osy) (17 gsm to about 204 gsm (grams per square meter)). In aspects where the coating composition is to be applied to a nonwoven fabric having a smaller fiber size, smaller void size and moderate level of breathability, smaller nanoparticles may be suitable. For example, nanoparticles having an average diameter of less than 100 nanometers may be suitable for use in a nonwoven fabric that includes a meltblown layer having a basis weight in the range of about 0.2 to about 1.0 osy (6.8 gsm to about 33.9 gsm).

While many different particles are useful in the present invention, silica particles may be particularly suitable for use in the present invention. Additionally, titanium dioxide, alumina, calcium carbonate, zeolite, laponite, magnesium oxide, carbon, copper, silver, polypropylene, polystyrene, and polylactic acid and other particles may also be used in the present invention. Further, the particles may be selected to provide a cooling or a heating effect. The particles in the coating composition can be of any general shape, and may have shapes such as an oblate or prolate spheroid, ovoid, discs, cylindrical or irregular shapes such as flakes and string-of-pearls.

To investigate the optimum ratio of components, experiments were conducted which varied the amount of aminofunctionalized silane to dialdehyde, and particle to aminofunctionalized silane to dialdehyde. Initial experiments indicated that, while different ratios of components performed effectively, particular ratios demonstrated a somewhat improved performance. More detailed experiments were conducted to evaluate these particular ratios and a desirable manner in which the components could be combined. The results of these more detailed experiments are reported in Table 1. For example, Table 1 delineates the weight ratios of the particle, aminofunctionalized silane and dialdehyde as well as the particle type and size. The average puncture resistance is provided, as well as the standard deviation. The puncture resistance of the samples in the experiments is representative of the rigidity/stiffness of the samples (i.e. the higher the puncture resistance, the more stiff the nonwoven fabric sample is).

Although different nonwoven fabrics may be used in the wearable articles of the present invention, all examples in Table 1 were created using the same nonwoven fabric, which is identified in Table 1 as “Base”. This base nonwoven fabric is an SMS laminate which is available from Kimberly-Clark Corporation as KIMGUARD KC400 wrap. In each test, a single sheet of 31 gsm SMS was utilized, as opposed to two sheets of SMS adhered together.

TABLE 1

| | | | | | | Ave. | Increase in |
|---|---|---|---|---|---|---|---|
| | | P:Sil:DiAld | | | | Puncture | Average |
| | Reaction | Weight | Particle Type & | | | Resistance | Puncture |
| No. | Sequence | Ratio | Size (nm) | | Silane | in Newtons | Resistance |
| Base | n/a | n/a | n/a | n/a | n/a | 1489 | n/a |
| 1 | Pre | 1:0.25:4 | silica | 15 | APTES | 2131 | 43% |
| 2 | n/a | 0.25:4 | none | n/a | APTES | 2599 | 75% |
| 3 | Post | 1:0.25:4 | silica | 15 | APTES | 2092 | 41% |
| 4 | 50-50 | 1:0.25:4 | silica | 15 | APTES | 1846 | 24% |
| 5 | 50-50 | 2:0.25:4 | silica | 15 | APTES | 1942 | 30% |
| 6 | 50-50 | 1:0.25:4 | silica | 15/400 | APTES | 1876 | 26% |
| 7 | Pre | 1:0.25:8 | silica | 15 | APTES | 2417 | 62% |
| 8 | Pre | 1:1:4 | silica | 15 | APTES | 2312 | 55% |
| 9 | Pre | 1:0.25:4 | silica | 55 | APTES | 2187 | 47% |
| 10 | Post | 1:0.25:4 | silica | 55 | APTES | 2452 | 65% |
| 11 | 50-50 | 1:0.25:4 | silica | 55 | APTES | 2046 | 37% |
| 12 | 50-50 | 2:0.25:4 | silica | 55 | APTES | 1772 | 19% |
| 13 | Pre | 1:0.25:8 | silica | 55 | APTES | 1810 | 22% |
| 14 | Pre | 1:1:4 | silica | 55 | APTES | 1916 | 29% |
| 15 | Pre | 1:0.25:4 | silica | 400 | APTES | 1730 | 16% |
| 16 | Post | 1:0.25:4 | silica | 400 | APTES | 1693 | 14% |
| 17 | 50-50 | 1:0.25:4 | silica | 400 | APTES | 1750 | 18% |
| 18 | 50-50 | 2:0.25:4 | silica | 400 | APTES | 2737 | 84% |
| 19 | 50-50 | 1:0.25:4 | silica | 400/15 | APTES | 1791 | 20% |
| 20 | Pre | 1:0.25:8 | silica | 400 | APTES | 1889 | 27% |
| 21 | Pre | 1:1:4 | Silica | 400 | APTES | 1830 | 23% |
| 22 | Pre | 1:0.25:4 | silica | 15 | TEOS | 2310 | 55% |
| 23 | n/a | 0.25:4 | none | n/a | TEOS | 1824 | 22% |
| 24 | Post | 1:0.25:4 | silica | 15 | TEOS | 2141 | 44% |
| 25 | Post | 1:0.25:4 | silica | 15 | HMDS | 2484 | 67% |
| 26 | Post | 1:0.25:4 | TiO2 | 25 | APTES | 2019 | 36% |
| 27 | Pre | 1:0.25:4 | TiO2 | 25 | APTES | 2568 | 72% |
| 28 | Post | 1:0.25:4 | alumina | 50 | APTES | 2541 | 71% |
| 29 | Pre | 1:0.25:4 | alumina | 50 | APTES | 1893 | 27% |

Puncture testing is commonly used to determine the strength of a material, and was conducted to determine the increase in average puncture resistance that the coating compositions of the invention may provide. Although there are numerous ways to perform puncture testing, the samples of Table 1 were subjected to the following test protocol. A constant rate of extension tensile tester was utilized in combination with a load cell that permits the peak load results to fall between about 10% and about 90% of the capacity of the load cell. The extension tensile tester utilized was the MTS 810, available from MTS Systems Corporation (Research Triangle Park, N.C.). Suitable load cells may be obtained from Instron Corporation (Canton, Mass.) or MTS Systems Corporation or another suitable vendor. A blade having a substantially flat edge was positioned perpendicular to the plane of the nonwoven fabric sample to be tested, and at an angle of 45 degrees with respect to the machine direction of the nonwoven fabric. As used herein, the terms “machine direction” or “MD” generally refers to the direction in which a material is produced. The term “cross-machine direction” or “CD” refers to the direction perpendicular to the machine direction. The cross-section of the blade which was utilized to puncture the nonwoven fabric had a thickness of 2 mm and a length of 30 mm. The height of the blade (that is, the length of the blade extending upwardly from the fabric) was 20 mm. Testing software, such as, for example, TESTWORKS software (available from MTS), is suitable for determining the required values.

Other tensile tester parameters included a cross-head speed of 800 inches per minute, a break sensitivity of twenty percent, and slack compensation of 10 grams-force. A test specimen of at least about 152.4 mm by 152.4 mm (6 inches by 6 inches) was positioned within the tester and clamped in place using a round circular rubber ring having a diameter of four inches (10 cm). About 20 psi was applied to the circular ring to hold the test specimen in place. For each example, three samples were prepared and tested for puncture resistance. The average of the maximum tensile force for the three samples was calculated and is shown in Table 1 as the Average Puncture Resistance.

For the purposes of the present invention, the average puncture resistance of all samples measured should show an increase over the average puncture resistance of the base nonwoven fabric. It is not required that the puncture resistance of every individual sample evaluated be greater than the base nonwoven fabric. The base sample was subjected to puncture resistance testing and had an average puncture resistance (peak load) of 335 lb-f (1491 N). The percent increase in average puncture resistance for all samples is reported in Table 1 and was calculated by subtracting from the average puncture resistance of the sample the average puncture resistance of the base nonwoven fabric (1489 N), multiplying by 100 and dividing by the average puncture resistance of the base nonwoven fabric (1489 N). A unique and unexpected result of the nonwoven fabrics of the present invention is the change in the sound that is made when the blade punctures the materials of the examples, even though all examples have an initial state of being flexible, drapable and breathable. In each of the samples of the present invention shown in Table 1, a distinct “pop” was heard when the blade penetrated the sample. This sound was not heard on the base control sample. Without wishing to be bound to any particular theory, it is believed that the loud “pop” is caused by the coating composition on the nonwoven fabric being able to absorb more energy prior to a catastrophic break. The opening formed in the nonwoven fabric having the coating composition is a clean cut. In contrast, the opening formed in the base nonwoven fabric is fuzzy. It is believed that the base nonwoven fabric opening is formed by the elongation of individual fibers before failure.

The examples also investigate when adding particles during the preparation of the coating composition provides a benefit. Specifically, experiments were conducted where particles were added at the beginning of the reaction (“Pre”), at the end of the reaction (“Post”), and where half the particles were added at the beginning and half the particles added at the end of the reaction (50-50). While not wishing to be held to a particular theory, it is believed that when the particles are added to the aminofunctionalized silane and dialdehyde mixture at the beginning of the reaction, the particles appear to be better incorporated into the composition. When the particles are added after the reaction of the aminofunctionalized silane and dialdehyde, it is thought that the particles link the ends of the aminofunctionalized silane/dialdehyde mixture into a network having some cross-linking. This cross-linking may occur at the beginning of the reaction or at the end of the reaction if the particles are sufficiently small to diffuse into the gel.

Looking at example 1 as described in Table 1, the coating composition that was applied to the SMS material was a 1:0.25:4 weight ratio of 15 nm silica particles, APTES and glutaraldehyde, respectively. To produce example 1, 0.25 grams of APTES and 20 ml of ethanol were stirred in a 50 ml round bottomed flask with a magnetic stir bar at room temperature for about 20 minutes. This solution was then poured into one gram of silica nanoparticles and the mixture was stirred for 20 minutes at ambient temperature. The mixture was then added to 20 ml of a 50% by weight solution of glutaraldehyde in deionized water and stirred at room temperature for about 60 minutes. This reaction sequence is referred to as “Pre” in Table 1.

Each of three 6 inch by 6 inch squares of SMS was separately placed into this mixture and permitted to soak for at least one to about ten seconds. The square of SMS was then passed through an Atlas Laboratory Wringer (model number LW-824, which is available from the Atlas Electric Company, Chicago Ill.) at a nip pressure of 6.8 kg and at the wringer's standard speed. Each square of SMS was air-dried in a fume hood at ambient temperature for at least about five hours and then subjected to puncture testing according to the methodology described above. The coating increased the average puncture resistance of the base fabric by 43%.

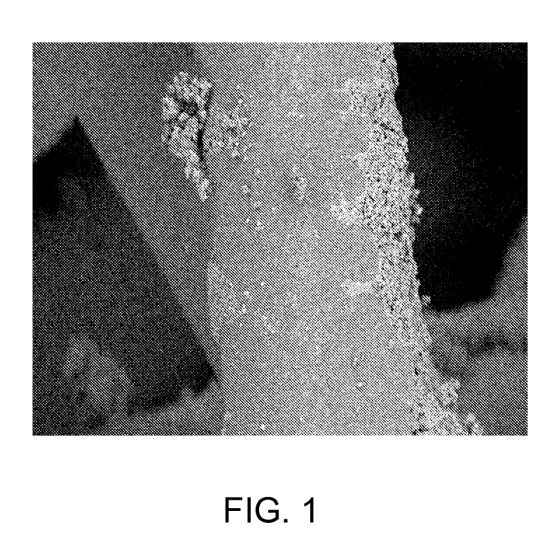

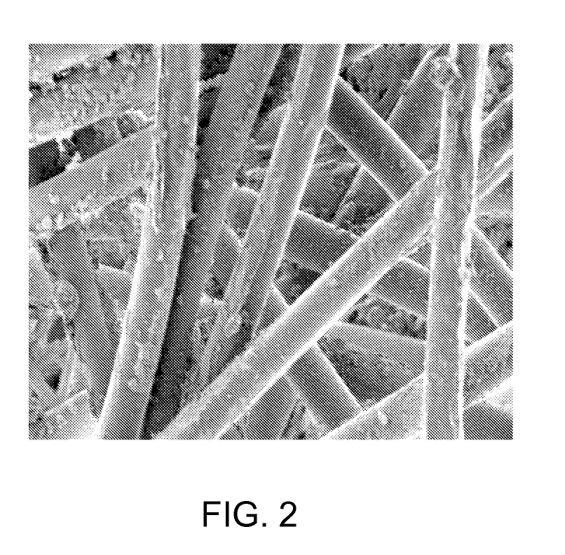

A coated fiber of an aspect of the present invention is shown in the photomicrograph of FIG. 1. While not all fibers are required to be fully coated, all the visible exterior surface area of the fiber shown in FIG. 1 is coated and additional coating is adhered to the fiber in clumps. FIG. 2 shows a plurality of such fibers in a nonwoven web, and demonstrates that the coating composition permits the nonwoven fabric to retain a significant portion of its original breathability by adhering to fibers rather than filling in the interstices in the nonwoven web. In preferred aspects, at least 50% of the fiber is coated with the coating composition, although other aspects may include fibers which have at least 60% of their exterior surface coated with the coating composition. Still other aspects may include fibers having at least 75% of their exterior surface coated with the coating composition, or in particular aspects may have at least 90% of their exterior surface coated. It is not necessary that the entire exterior surface of the fiber be coated with the coating composition, as synergies may be obtained by the mere layering of fibers in the nonwoven web. Similarly, additional synergies may be obtained by the layering of one or more nonwovens which have been treated with the coating composition of the present invention.

To approximate the percentage area of the fiber which is available or free of coating composition from a photomicrograph, the bright areas of the backscattered electron image are detected and isolated so that the total exposed area of the particles can be measured. An outline may be created which estimates the perimeter of the entire fiber, some of which may be covered by the coating composition. Standard image analysis software, such as IMIX by Princeton Gamma Tech, may be used to calculate the areas and determine the percent area of the visible exterior surface of the fiber which is coated by coating composition by dividing the area of the fiber which is coated with the coating composition by the estimated area of the fiber and multiplying by 100. While this process is inexact, it can provide a rough estimate of the percent area of the fiber which is coated with the coating composition.

In example 2, APTES was added to glutaraldehyde in a 0.25:4, ratio using the mixing, application and testing methodology described above, without the addition of particles. The increase in average puncture resistance was 75%. This example demonstrates that glutaraldehyde and APTES alone may form a sufficiently strong bond to improve the average puncture resistance of the base nonwoven fabric. Similarly, TEOS was added to glutaraldehyde in a ratio of 0.25:4 by weight (example 23) and provided an increase in average puncture resistance of 22%. While not wishing to be held to a particular theory, the substantial difference in average puncture resistance between these two examples may indicate that aminofunctional silanes may provide a greater improvement in the average puncture resistance than other silanes.

The coating composition of example 3 was prepared using a 1:0.25:4 weight ratio of silica particles having an average diameter of about 15 nm, APTES and glutaraldehyde. While the process of producing the exemplary coating composition described above is similar to the process by which example 3 was prepared, it is of note that the nanoparticles were added “post”, that is, after the APTES and glutaraldehyde were combined. The increase in average puncture resistance was 41%.

Example 4 was prepared using a ratio of 1:0.25:4 by weight of 15 nm silica particles, APTES and glutaraldehyde. Half of the silica nanoparticles were added at the beginning of the reaction (as in the “Pre” reaction sequence of example 1) and half of the silica nanoparticles were added at the end of the reaction (as in the “Post” reaction sequence of example 3). This reaction sequence has been designated “50-50” in Table 1, indicating that 50% of the particles by weight were added during the reaction sequence and 50% of the particles by weight were added at the end of the reaction sequence. The increase in average puncture resistance for example 4 was 24%. Similarly, example 5 was prepared using a 50-50 process with silica particles having an average diameter of about 15 nm, APTES and glutaraldehyde in a ratio by weight of 2:0.25:4, respectively. The increase in average puncture resistance was 30%.

Example 6 was prepared using a ratio of 1:0.25:4 by weight of silica particles, APTES and glutaraldehyde. Half of the silica nanoparticles by weight had an average diameter of 15 nm, and these nanoparticles were added at the beginning of the reaction. The remaining half of the silica nanoparticles by weight had an average diameter of 400 nm, and these nanoparticles were added at the end of the reaction. The increase in average puncture resistance was 26%. Similarly, example 19 also utilized silica nanoparticles in which half of the nanoparticles by weight had an average diameter of 400 nm and the remaining nanoparticles had an average diameter of 15 nm. In example 19, the 400 nm silica nanoparticles were added earlier in the process while the 15 nm silica nanoparticles were added at the end of the process. The coating of example 19 increased the average puncture resistance of the SMS by 20%.

In examples 7 and 8, the 15 nm silica particles were added to APTES and glutaraldehyde in the same manner as was used for example 1. In contrast to example 1, the weight ratio for example 7 was 1:0.25:8 and 1:1:4 for example 8. The increase in average puncture resistance provided by examples 7 and 8 were 62% and 55%, respectively.

Examples 9 through 14 were prepared using APTES, glutaraldehyde and silica particles having an average diameter of about 55 nm, although the reaction sequence and weight ratios for the examples varied. The increase in average puncture resistance varied from 19% to 65% for these samples. From these examples, the increase in size of the nanoparticles from 15 to 55 nm did not appear to impact the function of the coating composition on the SMS material. It is possible that, for other substrates, a similar increase in size of the nanoparticles may impact the increase in average puncture resistance obtained.

Examples 15 through 18, 20 and 21 were formed from APTES, glutaraldehyde and 400 nm silica particles, with varying reaction sequences and weight ratios. The increase in average puncture resistance varied from 14% to 84%. This level of variation may be due in part to the size of the silica nanoparticles with respect to the voids in the meltblown layer of the SMS material.

Examples 22, 24 and 25 investigate the use of silica nanoparticles with TEOS and HMDS rather than APTES. In examples 22 and 24, the coating composition with TEOS and nanoparticles functioned well by providing increases in average puncture resistance of 55% and 44%, respectively. Example 25 utilized HMDS as the aminofunctionalized silane, and increased the average puncture resistance of the base material by 67%.

Examples 26 and 27 evaluated the use of titanium dioxide as the nanoparticle of the composition, with increases in average puncture resistance of 36% and 72%. Similarly, examples 28 and 29 evaluated the use of alumina as the nanoparticle of the composition, with increases in average puncture resistance of 71% and 27%. The examples shown demonstrate that the coating composition of the present invention is able to increase the average puncture resistance of a nonwoven fabric.

Further tests showing the benefits of the nonwoven fabric of the invention were conducted on samples representative of Example 25 and on samples of base nonwoven fabric that were not treated with the coating composition of the invention. The tests included Taber Abrasion Test (Table 2), sliding compression test (toughness) (Table 3) and linting test (torsion test to determine coating durability) (Table 4).

Samples of the base nonwoven fabric and samples representative of Example 25 were submitted for Taber Abrasion testing (using Standard Test Method 2204 dated Nov. 23, 2010 test type method A). The results (average of two samples) are shown in Table 2. The higher Final Rating for the Example 25 samples shows that the nonwoven fabrics of the invention have higher resistance to abrasion than the base nonwoven fabric.

TABLE 2

| Sample | Taber Abrasion Final Rating |
|---|---|
| Base | 2 |
| 25 | 4 |

Samples of the base nonwoven fabric and samples representative of Example 25 were also submitted for Sliding Compression testing (using Standard Test Method 4566 dated Sep. 29, 2009). The results (average of three samples) are shown in Table 3. The higher Sliding Compression value for the Example 25 samples shows that the nonwoven fabrics of the invention have improved toughness compared to the base nonwoven fabric.

TABLE 3

| | Sliding Compression |
|---|---|
| Sample | Wf in grams |
| Base | 867 (STD 64) |
| 25 | 1150 (STD 108) |

Samples of the base nononwoven fabric and samples representative of Example 25 were also submitted for resistance to linting testing. The amount of lint for a given sample was determined according to the Gelbo Lint Test. The Gelbo Lint Test determines the relative number of particles released from a fabric when it is subjected to a continuous flexing and twisting movement. It is performed in accordance with INDA test method 160.1-92. A 9 inch by 9 inch square sample is placed in a flexing chamber. As the sample is flexed, air is withdrawn from the chamber at 1 cubic foot per minute for counting in a laser particle counter. The particle counter counts the particles by size using channels to size the particles. The results (average of three samples) are reported as an average of the average number of particles counted in the ten counting periods for each particle size range.

TABLE 4

| | No. | | | | | |
|---|---|---|---|---|---|---|
| | >0.3 | >0.5 | >1 | >5 | >10 | >25 |
| | micron | micron | micron | micron | micron | micron |
| Base | 1101.8 | 589.4 | 78.9 | 10.5 | 8.4 | 2.0 |
| 25 | −96.8 | −51.9 | −13.3 | −5.4 | −4.1 | −2.0 |

As shown in Table 4, the Example 25 samples have a coating that is very durable, giving rise to no detectable linting or dusting. The results show that the base nonwoven fabric has higher linting that the nonwoven fabric coated with the composition of Example 25. The results for the Example 25 samples are negative (showing an improvement) because the results for the base nonwoven fabric were subtracted from the results for the Example 25 samples for each size range.

In order to show the change in mechanical properties of the nonwoven fabric between an initial state and a final state after the nonwoven fabric experiences a sudden force, a different set of experiments were conducted. In this set, nonwoven fabric samples that were treated with the coating composition of the invention were evaluated for their total energy to compress. The nonwoven fabric samples were treated with the composition of Example 25 from Table 1 above; Example 25 had a ratio of particles to aminofunctionalized silane to dialdehyde of 1:0.25:4, the particles were silica and 15 nm in size and the silane was HMDS. The change in mechanical properties was measured by determining the Edge-wise Compression (EC) value as follows: a 2 inch by 6 inch (5.1 cm×15.24 cm) piece of nonwoven fabric was cut with its longer dimension aligned with the longitudinal direction of the nonwoven fabric web. The weight of the samples was determined. The thickness of the samples was determined under a 0.2 psi (1.38 KPa) load. The sample of nonwoven fabric was formed into a cylinder having a height of 2 inches (5.1 cm), and with the two ends having 0-0.125 inch (0-3.18 mm) overlap, the nonwoven fabric was stapled together with three staples. One staple was near the middle of the width of the sample, the other two nearer each edge of the width of the sample. The longest dimension of the staple was in the circumference of the formed cylinder to minimize the effect of the staples on the testing. An INSTRON tester, or similar instrument, was configured with a bottom platform, a platen larger than the circumference of the sample to be tested and parallel to the bottom platform, attached to a compression load cell placed in the inverted position. The sample was placed on the platform, under the platen. The platen was brought into contact with the sample and compressed the sample at a rate of 400 inches/min. and 200 inches/min. The energy required to compress the sample to 50% of its width (1 inch) (2.54 cm) was recorded. The results are provided in Table 5 below.

TABLE 5

| | | Specimen | Specimen | Energy | Basis | |
|---|---|---|---|---|---|---|
| Sample | | Thickness | Weight | to 50% | Weight | Density |
| I.D. | Spcmn No. | mm | Gf | gf * mm | gf/m\hat{ }2 | g/cm\hat{ }3 |
| #25 @ 400 in/min | 1 | 0.52 | 0.47 | 5894.63 | 30.35 | 0.06 |
| | 2 | 0.47 | 0.48 | 6509.98 | 31.00 | 0.07 |
| | 3 | 0.50 | 0.56 | 6750.72 | 36.17 | 0.07 |
| | Ave | 0.50 | 0.50 | 6385.11 | 32.51 | 0.07 |
| | Std | 0.03 | 0.05 | 441.49 | 3.19 | 0.01 |
| #25 @ 200 in/min | 1 | 0.61 | 0.49 | 5014.89 | 31.65 | 0.05 |
| | 2 | 0.65 | 0.56 | 5379.87 | 36.17 | 0.06 |
| | 3 | 0.52 | 0.51 | 3773.80 | 32.94 | 0.06 |
| | Ave | 0.59 | 0.52 | 4722.85 | 33.59 | 0.06 |
| | Std | 0.07 | 0.04 | 841.92 | 2.33 | 0.01 |

The results in Table 5 show that the nonwoven fabric samples made of a plurality of fibers coated with the coating composition of the invention are flexible and drapable in an initial state. The total energy to compress to 50% is at least 30% greater at a compression rate of 400 inches/minute compared to the total energy to compress to 50% at a compression rate of 200 inches/minute as measured by the EC test. The average energy to compress of the samples at a compression rate of 400 inches/minute was 6385.11 gf*mm while the average energy to compress of the samples at a compression rate of 200 inches/minute was 4722.85 gf*mm. The mechanical properties of the samples change when subjected to a sudden force; the samples would no longer be flexible and drapable and instead would become stiff and rigid. The specific mechanical properties would depend on the construction of the samples (e.g. number of layers), the basis weight of the nonwoven fabric(s) and the amount and type of coating composition applied to the fibers of the nonwoven fabric(s).

The wearable articles of the invention have a generally planar surface that may include a thermal change zone. The thermal change zone provides the capability of additional therapeutic benefits through the delivery of either cold or heat when the wearable article experiences a sudden force. Delivery of a temperature change to make the affected body part cold could have the benefit of reducing swelling, bruising or acute pain. Delivery of a temperature change to make the affected body part experience heat could have the benefit of stimulating circulation and alleviating soreness or muscle stiffness. A cooling temperature change can occur through the mixing of thermal reagents like urea, ammonium nitrite, xylitol, sorbitol, mannitol or similar reagents with water. A heating temperature change can occur through exposure of thermal reagents like iron oxidation chemistry components or magnesium oxide with air; or alternatively, the heat released by nucleation of a sodium acetate and water solution to cause crystallization.

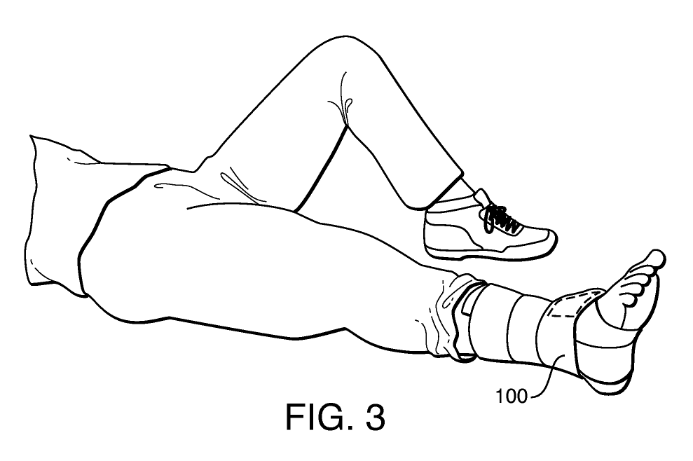

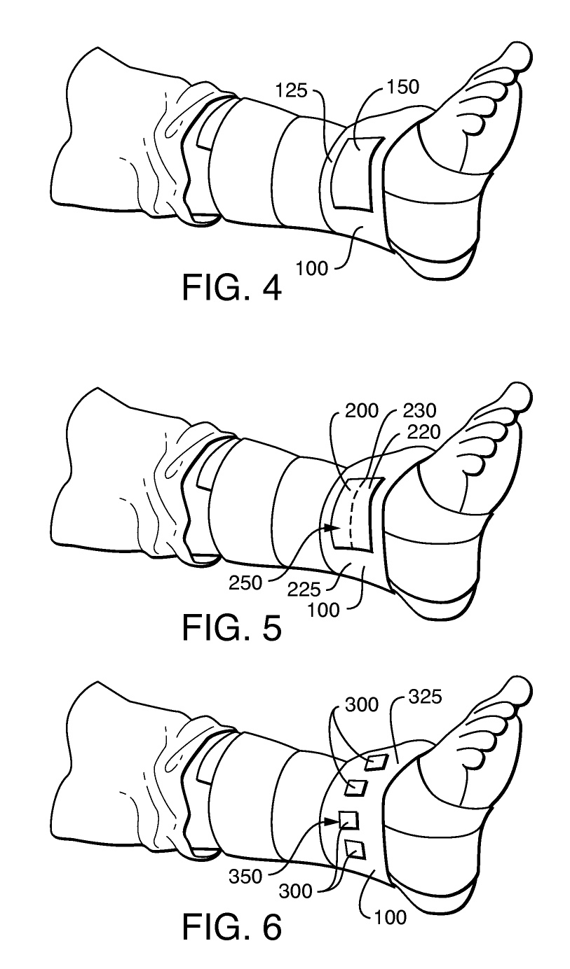

FIG. 3 representatively shows a wearable article 100 of the present invention being worn to protect the ankle of the user. The person represented in FIG. 3 is shown after an unexpected fall caused a sudden force acting on their ankle. The wearable article 100 desirably reacts to the sudden force by increasing in stiffness and immobilizing the affected area to reduce further injury/trauma. FIG. 4 representatively illustrates a wearable article 100 of the invention being worn around the ankle of the user. The wearable article 100 has a planar surface 125 that includes a thermal change zone 150. The thermal change zone 150 may include a planar reservoir that contains a thermal reagent. The thermal reagent is capable of generating heat through an exothermic reaction when the thermal reagent is exposed to air. The thermal change zone 150 may also include a material to retain the generated heat such as calcium carbonate particles, ceramic beads, phase change materials such as waxes and other similar materials. The heat may provide a therapeutic benefit to the injured ankle if the user is experiencing soreness or stiffness. Alternatively, the thermal reagent may be capable of generating cold through an endothermic reaction when the thermal reagent is exposed to air. The cold may provide a therapeutic benefit to the injured ankle if the user is experiencing or is at risk of swelling, bruising or acute pain. FIG. 5 representatively illustrates another aspect of the present invention and depicts a wearable article 100 being worn around the ankle of a user. The wearable article 100 has a planar surface 225 that includes a thermal change zone 250. The thermal change zone 250 includes two, planar reservoirs, 200 and 220, that are separated by a frangible seal 230. The thermal reagents contained in the planar reservoirs, 200 and 220, may be selected from proton-contributing materials and proton-accepting materials. When the wearable article 100 is exposed to a sudden force, the frangible seal 230 will rupture and the contents of the planar reservoirs, 200 and 220, will mix to result in a heat-producing exothermic reaction or a cold-producing endothermic reaction. In another aspect, FIG. 6 representatively illustrates a wearable article 100 of the invention also being worn around the ankle of a user. The wearable article 100 has a planar surface 325 that includes a thermal change zone 350. The thermal change zone 350 includes several, planar reservoirs 300, each reservoir 300 containing a thermal reagent. The reservoirs 300 may be capable of rupturing when the wearable article 100 experiences a sudden force. The thermal reagents contained in the planar reservoirs 300 may be capable of generating heat or cold through exothermic or endothermic reactions. The thermal change may provide a therapeutic benefit to the injured ankle.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A wearable article comprising a nonwoven fabric that includes a plurality of coated fibers, each coated fiber comprising:
a fiber having an exterior surface; and
a coating composition disposed on at least a portion of the exterior surface of the fiber, the coating composition comprising an aminofunctionalized silane and a dialdehyde, wherein the weight percent of the dialdehyde in the coating composition is greater than the weight percent of the silane in the coating composition.

2. The wearable article of claim 1, wherein the wearable article is selected from wraps, braces, supports, compression hosiery, bandages and compresses.

3. The wearable article of claim 1, wherein the nonwoven fabric is flexible and requires a total energy to compress that is at least 25% greater at a compression rate of 400 inches/minute compared to a total energy to compress at 200 inches/minute as measured by a ring compression test.

4. The wearable article of claim 1, wherein the wearable article further comprises a film material.

5. The wearable article of claim 1, wherein the nonwoven fabric is a laminate that includes a spunbond layer and a meltblown layer.

6. The wearable article of claim 1, wherein the dialdehyde is glutaraldehyde.

7. The wearable article of claim 1, wherein the weight percent of the dialdehyde is at least twice the weight percent of the aminofunctionalized silane in the coating composition.

8. The wearable article of claim 1, wherein the aminofunctionalized silane is aminopropyltriethoxysilane or hexamethyldisilazane.

9. The wearable article of claim 1, wherein at least 50% of the exterior surface of the fiber is coated with the coating composition.

10. The wearable article of claim 1, wherein the fiber is corona treated.

11. The wearable article of claim 1, wherein the wearable article includes a planar surface, wherein the planar surface comprises a thermal change zone.

12. The wearable article of claim 11, wherein the thermal change zone includes two, planar reservoirs, each reservoir containing a thermal reagent and the planar reservoirs separated by a frangible seal.

13. The wearable article of claim 11, wherein the thermal change zone includes a planar reservoir containing a thermal reagent and wherein the planar reservoir includes a frangible seal.

14. The wearable article of claim 12, wherein the thermal reagent is selected from a proton-contributing material and a proton-accepting material.

15. A wearable article comprising a nonwoven fabric that includes a plurality of coated fibers, each coated fiber comprising:
a fiber having an exterior surface; and
a coating composition disposed on at least a portion of the exterior surface of the fiber, the coating composition comprising particles, an aminofunctionalized silane and a dialdehyde, wherein the weight percent of dialdehyde in the coating composition is greater than the weight percent of aminofunctionalized silane in the coating composition.

16. The wearable article of claim 15, wherein the particles are selected from silica, titanium dioxide, calcium carbonate, alumina, zinc oxide, clay and metal hydride salts.

17. The wearable article of claim 15, wherein the particles have an average particle size of less than 250 nanometers.

18. The wearable article of claim 15, wherein the particles have an average particle size of less than 150 nanometers.

19. The wearable article of claim 15, wherein the weight percent of dialdehyde in the coating composition is at least twice the weight percent of particles in the coating composition.

20. A method of protecting a body part that experiences a sudden force or impact comprising the steps of:
providing a wearable article formed of a nonwoven fabric comprising fibers, wherein the fibers have an exterior surface and include a coating composition, wherein the coating composition comprises an aminofunctionalized silane and dialdehyde, wherein the weight percent of the dialdehyde in the coating composition is greater than the weight percent of the aminofunctionalized silane in the coating composition; and
donning the wearable article on the body part to be protected.
